(19) United States
(12) Patent Application Publication
(10) Pub. No.: US 2004/0005310 A1
(43) Pub. Date: Jan. 8, 2004

(54) Method for reconstituting lyophilized proteins

(76) Inventors: Mirna Rapp, Marburg (DE); Michel Grandgeorge, Vaugneray (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/384,748

(22) Filed: Mar. 11, 2003

(30) Foreign Application Priority Data
Mar. 13, 2002 (DE) 10211227.4

(51) Int. Cl. A61K 38/37; A61K 38/48

(52) U.S. Cl. 424/94.63; 514/12

(57) ABSTRACT

A method for reconstituting lyophilized proteins, in which the reconstitution takes place in a receptacle in which the gas pressure is between 1 mbar and atmospheric pressure, is described. In the lyophilized protein-containing receptacle, the gas pressure of between 1 mbar and atmospheric pressure, which gas pressure is required in accordance with the invention, is set by introducing air or an inert gas, and the quantity of water for injection purposes which is required for dissolving the protein is added. The method is particularly suitable for blood coagulation factors, the von Willebrand factor and albumin.

The invention relates to a method for reconstituting lyophilized proteins in which optimal dissolution behavior of the protein is ensured by setting defined pressure ratios during reconstitution.

It is customary to use a freeze drying, which is termed lyophilization, to convert proteins which are used as pharmaceuticals into a form which can be stored over a relatively long period of time and, in connection with this, preserve their biological activity. In freeze drying, the labile proteins are frozen rapidly and carefully under sterile conditions, after which the water, which has been converted into ice, is rapidly removed by sublimation under high vacuum conditions. At the same time, the substance which is to be dried remains in the frozen state due to the cold arising from the evaporation.

When lyophilizates are prepared in practice, the solutions containing the protein are customarily aliquoted into small receptacles which are designed for the vapor release, after which what are termed lyophilization stoppers are put on them and they are introduced into a freeze drying chamber. While still on the shelves, the receptacles are then sealed in the freeze drying chamber by the shelves being pressed together hydraulically or manually, with the stoppers being pressed down into the receptacles. This ensures that the vacuum in the receptacles is comparable with that in the freeze drying chamber. Subsequently, the chamber is ventilated, and the receptacles, containing the finished lyophilizates, are removed and subsequently sealed with flange caps and packed ready for use.

This method is used on a large scale, for example for the blood coagulation factors, including the factors VIII, von Willebrand factor (vWF) and factor IX (F IX). For stabilization purposes, the lyophilizates can contain salts, monosaccharides, polysaccharides, organic polymers and other proteins, such as albumin. The lyophilized proteins can be administered, either on their own or combined in finished products, for treating hemophilia A or von Willebrand disease, for example. Combinations of the blood coagulation factor VIII and the von Willebrand factor can be used for treating von Willebrand disease without any additional administration of factor VIII. In the case of treating hemophilia A, a relatively large proportion of von Willebrand factor was found to be advantageous for protecting against the possible formation of inhibitors of blood coagulation factor VIII. After a mixture of the two components has been prepared, such a combination preparation is aliquoted into small glass bottles and lyophilized in accordance with a defined program. After the freeze drying has taken place, the lyophilizate is sealed while still under vacuum. The presence of a negative pressure in the receptacle is a prerequisite for the success of the dissolution process which takes place directly before the active compound solution is drawn up into the syringe. In this connection, the lyophilizate is dissolved in a physiologically tolerated aqueous solvent, for example PBS (phosphate-buffered saline) or water for injection purposes (WFI), which is contained in a separate glass vessel. A carry-over system is used to transfer the solvent into the receptacle containing the lyophilizate. This ensures that the solvent flows in rapidly under conditions which are as germ-free as possible.

It has now been observed that, while a very low value for the vacuum ensures that the WFI flows rapidly into the receptacle, such a low value can have a disadvantageous effect on the solubility of the lyophilizate. A consequence of the negative pressure which prevails in the pharmaceutical bottle being too great can be that the dissolution of small amounts of the lyophilizate can be delayed to such an extent that the period envisaged for completely dissolving the protein is exceeded. Even though almost the whole amount of the lyophilizate dissolves rapidly, remnants of the lyophilizate, which float on the surface of the aqueous solution, nevertheless still remain in a number of cases. These remnants are very light and cannot even be sedimented by centrifugation at up to 14 000 rpm. Viewed microscopically, they contain many tiny enclosed air bubbles. These bubbles are probably a consequence of air or water vapor inclusions being formed during the reconstitution under a very high vacuum. The dissolution of these difficultly soluble lyophilizate remnants is delayed to such an extent that in some cases up to 4 hours elapse before dissolution is complete. Complete dissolution is regarded as being the absence of optically discernible unsedimentable particles. Biochemical analysis shows that these difficultly soluble particles have a chemical composition which is identical to that of the dissolved material and do not contain any other foreign particles or degradation products either. While the difficultly soluble lyophilizate remnants are on average 2 mm in size, they do not represent any danger to the patient since the solution which is prepared in the receptacle is always conducted through a filter system which is contained in the pack, resulting in undissolved constituents being removed. This also applies to other undissolved constituents, i.e. what are termed fusel form particles, which are microscopically compact, and therefore sedimentable particles which are occasionally observed after von Willebrand factor-containing preparations have been dissolved.

The object of improving the method for reconstituting lyophilized proteins and achieving rapid and complete dissolution therefore presented itself. In this connection, the point of departure for the solution which was finally found was the observation that the addition of water for injection purposes to a vacuum-free receptacle leads to complete dissolution of the entire lyophilizate cake within 1 to 2 minutes. This led to the recognition of there being a connection between the vacuum in the lyophilizate-containing receptacle and the appearance of particles exhibiting delayed dissolution behavior. Further investigations have then shown that the gas pressure which prevails in the lyophilizate receptacle at the beginning of the reconstitution has a decisive influence on the dissolution of the lyophilized protein.

In these investigations, a method for reconstituting lyophilized proteins was found in which the reconstitution takes place in a receptacle in which the gas pressure is between 1 mbar and atmospheric pressure. A method in which the reconstitution takes place in a receptacle in which the gas pressure is between 100 mbar and atmospheric pressure is advantageous. A method in which the gas pressures [sic] is between 100 and 300 mbar is particularly advantageous. Under these conditions, the appearance of difficultly soluble particles (lyophilizate remnants) can be very reliably avoided. Rapid transfer of the solvent, that is the water for injection purposes, for example, into the lyophilizate receptacle can be effected just as well under the abovementioned pressure conditions as it can at vacuum values of less than 1 mbar.

The method according to the invention for reconstituting proteins requires that there should no longer be a high vacuum in the receptacle containing the lyophilizate. In general, a freeze drying is carried out such that the entire operation is performed under constant pressure and temperature conditions as soon as the freezing process has been completed. However, a freeze drying of sensitive materials, such as plasma proteins, is normally run in accordance with a precisely defined program in which the temperature of the shelves in the freeze drying chamber is raised stepwise after defined time intervals. In the same way, the vacuum conditions can be varied in order to maintain a desired residual moisture content in the lyophilizate after the freeze drying has been completed.

According to the invention, therefore, after the lyophilization has been completed under high vacuum, the gas pressure in the protein-containing receptacle is adjusted to between 1 mbar and atmospheric pressure, preferably between 100 mbar and atmospheric pressure, by introducing air or an inert gas, and the quantity of water for injection purposes which is required for dissolving the protein is added to the receptacle.

The adjustment to a defined vacuum value can be effected either by appropriately altering the program conditions or by ventilating the drying chamber from time to time. The ventilation can be effected by supplying air or supplying inert gases such as nitrogen. Since lyophilized products usually have to be sealed under sterile conditions, the gas which is supplied is conducted through a sterilizing filter.

In addition, the invention relates to a kit for preparing an injectable solution of at least one therapeutic protein, with the kit comprising a receptacle which contains the therapeutic protein(s) and in which the air pressure has been reduced to not less than 1 mbar, with it also being possible for the receptacle to be filled entirely or partially with a protective gas such as nitrogen. It is advantageous if the air pressure in the receptacle is between 100 and 300 mbar. The kit according to the invention can also additionally comprise a receptacle containing a physiological solvent. The therapeutic protein employed, which can, for example, be a blood coagulation factor, in particular factor VIII, can be of natural origin or can have been obtained recombinantly. It is likewise possible to use mutants or constituent sequences of blood coagulation factors as therapeutic proteins within the meaning of the invention.

The invention is explained in more detail in the following examples.

### EXAMPLE 1

The reconstitution of Haemate HS/Humate P (Aventis Behring, Marburg, Germany) using aliquots which were sealed under high vacuum was compared with the reconstitution of aliquots in which there was no vacuum (atmospheric pressure). The solvent (WFI) was transferred into the receptacle containing the lyophilizate using a carry-over system or using a disposable syringe. In every case, the result showed complete dissolution of the lyophilizate under atmospheric pressure. In most cases, aliquots under high vacuum exhibited a delay in the dissolution of small lyophilizate remnants of more than 10 minutes. The rate at which WFI was administered was of no consequence for the appearance of difficultly soluble lyophilizate remnants.

### EXAMPLE 2

Lyophilized aliquots of Haemate were sealed at different vacuum values in the freeze drying unit. The reconstitution with WFI was carried out using the carry-over system which is authorized for Haemate HS/Humate P. The time required for the WFI to flow in, and the appearance of difficultly soluble lyophilizate remnants, were recorded. As shown in table 1, a vacuum value of greater than 100 mbar is advantageous for complete reconstitution of the lyophilizate. It is still possible to transfer the solvent rapidly under these pressure conditions.

TABLE 1
Reconstitution behavior of Haemate HS/Humate P in the
aliquots having different vacuum values

| Vacuum in the | | Time taken for | Difficultly soluble |
|---|---|---|---|
| lyophilizate | | the solvent to | lyophilizate |
| receptacle | | flow in | remnants observed |
| 0.025 | mbar | 11 sec. | Yes |
| 0.25 | mbar | 13 sec. | Yes |
| 1 | mbar | 13 sec. | Yes |
| 100 | mbar | 13 sec. | Yes/No |
| 175 | mbar | 14 sec. | No |
| 250 | mbar | 14 sec. | No |

1. Method for reconstituting lyophilized proteins, characterized in that the reconstitution takes place in a receptacle in which the gas pressure is between 1 mbar and atmospheric pressure.

2. Method according to claim 1, characterized in that the reconstitution takes place in a receptacle in which the gas pressure is between 100 mbar and atmospheric pressure.

3. Method according to claims 1 and 2, characterized in that, after the lyophilization has been completed under high vacuum, the gas pressure in the protein-containing receptacle is adjusted to between 1 mbar and atmospheric pressure by introducing air or an inert gas, and the quantity of solvent which is required for dissolving the protein is added to the receptacle.

4. Method according to claim 3, characterized in that the air or the inert gas which is employed for adjusting the gas pressure in the receptacle is conducted through a sterilizing filter.

5. Method according to claims 1 to 4, characterized in that the protein employed is a plasma protein.

6. Method according to claims 1 to 5, characterized in that the plasma protein employed is a blood coagulation factor, the von Willebrand factor or albumin.

7. Method according to claims 1 and 2, characterized in that, after the lyophilization has been completed under high vacuum, the gas pressure in the protein-containing receptacle is adjusted to between 1 mbar and atmospheric pressure by appropriate choice of the program conditions in the freeze drying unit, and the quantity of water for injection purposes which is required for dissolving the protein is added to the receptacle.

8. Kit for preparing an injectable solution of at least one therapeutic protein, characterized in that it comprises a receptacle which contains the therapeutic protein(s) and in which the air pressure has been reduced to not less than 1 mbar.

9. Kit according to claim 8, in which the receptacle contains a protective gas.

10. Kit according to claim 8 or 9, in which the air pressure in the receptacle is between 100 and 300 mbar.

11. Kit according to one of claims 8 to 10, which additionally comprises a receptacle containing a physiological solvent.

12. Kit according to one of claims 8 to 11, in which the therapeutic protein is natural or recombinantly obtained factor VIII or a mutant of factor VIII.
